(19) United States
(12) Patent Application Publication
(10) Pub. No.: US 2016/0113967 A1
(43) Pub. Date: Apr. 28, 2016

(54) SYSTEMS AND METHODS FOR MANIPULATION OF REGENERATIVE CELLS FROM ADIPOSE TISSUE

(71) Applicant: Cytori Therapeutics, Inc., San Diego, CA (US)

(72) Inventors: Marc H. Hedrick, Encinitas, CA (US); John K. Fraser, San Diego, CA (US)

(21) Appl. No.: 14/860,416

(22) Filed: Sep. 21, 2015

Related U.S. Application Data
(63) Continuation of application No. 13/926,854, filed on Jun. 25, 2013, now abandoned.
Application No. 13/926,854 is a continuation of application No. 12/302,787, filed on Aug. 12, 2009, now abandoned.

(51) Int. Cl. A61K 35/28; C12N 5/0775; A61K 9/00

(52) U.S. Cl. CPC A61K 35/28; A61K 9/0024; C12N 5/0667; C12N 2509/10

(57) ABSTRACT

The invention provides methods for manipulating regenerative cells from adipose tissue. Specifically, it provides methods for enrichment of desired cells and enhancement of their therapeutic effects.

### CROSS-REFERENCE TO RELATED APPLICATIONS

This application is a continuation of, and claims the benefit of priority to, U.S. patent application Ser. No. 13/926,854, filed Jun. 25, 2013, which is a continuation of, U.S. patent application Ser. No. 12/302,787, filed Aug. 12, 2009, which is the U.S. national phase under 35 U.S.C. §371 of prior PCT International Application No. PCT/US2006/021017, filed May 30, 2006, each of which is incorporated by reference in its entirety.

### FIELD OF THE INVENTION

The present invention relates to the field of medicine, particularly, to methods for manipulating regenerative cells found in adipose tissue.

### BACKGROUND OF THE INVENTION

Regenerative cells from adipose tissue, and certain methods for separating them from tissue and other materials, as well as for concentrating them, have been described, e.g., in U.S. Pat. Pub. No. 2005/0084961, entitled “SYSTEMS AND METHODS FOR SEPARATING AND CONCENTRATING REGENERATIVE CELLS FROM TISSUE.” Further methods for optimizing and enhancing separation, concentration, and function of these cells would provide benefit to patients potentially treated with regenerative cells.

### SUMMARY OF THE INVENTION

Adipose derived regenerative cells, e.g., stem and progenitor cells, have been shown to confer a therapeutic, structural or cosmetic benefit in a wide variety of diseases and disorders. This therapeutic, structural or cosmetic benefit may be optimized or enhanced by manipulation of the regenerative cells at some point prior to placement into a recipient.

The present invention relates to methods and apparati for processing regenerative cells from adipose tissue comprising separating the regenerative cells from other sample components; concentrating the regenerative cells; and manipulating the regenerative cells; wherein said processing results in the removal of undesired sample components or fractionation of the regenerative cells.

In particular embodiments, the manipulating step comprises physical manipulation, including but not limited to exposure to hypoxic or hyperoxic conditions, mechanical stimulation, ultrasonic stimulation, electrical stimulation, temperature changes, exposure to infrared light, or exposure to UV lights. Other contemplated physical manipulations are separation by density gradient centrifugation or continuous flow centrifugation. In further embodiments, the physical manipulation comprises adhering sample components to a solid phase surface, wherein said adherence selects either the regenerative cells or other sample components to be separated from the regenerative cells. In specific embodiments, the solid phase surface is selected from the group comprising tissue culture plastic, plastic beads, glass beads, scaffolds or any combinations thereof.

The invention also relates to methods and apparati for processing regenerative cells from adipose tissue comprising: separating the regenerative cells; concentrating the regenerative cells; and manipulating the regenerative cells; wherein said processing results in an improvement in yield or viability of the regenerative cells. In embodiments, the manipulating step comprises exposing the regenerative cells to a chemical agent or additive. In other embodiments, the manipulating step comprises exposing the regenerative cells to a biological agent or additive.

Further, the invention includes methods for processing regenerative cells from adipose tissue comprising: separating the regenerative cells; concentrating the regenerative cells; manipulating the regenerative cells; wherein said processing results in an improved therapeutic benefit of the regenerative cells. In embodiments, the manipulating step comprises exposing the regenerative cells to a chemical agent or additive. In other embodiments, the manipulating step comprises exposing the regenerative cells to a biological agent or additive.

In embodiments, the regenerative cells processed according to any of the methods of the invention are delivered to a patient. In these embodiments, it is contemplated that the regenerative cells are delivered through scaffolds, carriers, injection, injectable solutions, beads, microspheres, nanospheres, hydrogels, gels, polymers, ceramics, collagen gels, platelet gels, hyaluronon based scaffolds, apatite coated scaffolds, self-assembled peptides, in combination with other cells or tissue or tissue fragments, timed release delivery devices or any combinations thereof.

### BRIEF DESCRIPTION OF THE DRAWINGS

FIG. 1 Exemplary System for Harvesting Cells from Adipose Tissue. The FIGURE shows an illustration of an exemplary system 10 for separating and concentrating regenerative cells from tissue. Parts of the system as represented in the FIGURE are: conduits 12; positive displacement pump on the conduit 12d; collection chamber 20; solution sources 22; washing solution source 23; tissue disaggregation agent source 24; prefixed filter 28; automated sensors 29; processing chamber 30; pump 34; waste container 40, and; sample chamber 60.

### DETAILED DESCRIPTION OF THE PREFERRED EMBODIMENT

The present invention relates to methods for manipulating regenerative cells, which can be separated and concentrated from adipose tissue using any of the methods described herein, incorporated by reference, or known in the art. The regenerative cells can be manipulated such that their function, viability, purity or any other feature is altered. In particular embodiments, one or more functions of the regenerative cells is enhanced. In a preferred embodiment, the manipulated regenerative cells are suitable for placement into a recipient.

The regenerative cells may be separated and concentrated by any of the methods described in, e.g., U.S. application Ser. No. 10/884,638 (U.S. Pub. No. 2005/0260175) entitled SYSTEMS AND METHODS FOR ISOLATING AND USING CLINICALLY SAFE ADIPOSE DERIVED REGENERATIVE CELLS, U.S. application Ser. No. 10/316,127, entitled SYSTEMS AND METHODS FOR TREATING PATIENTS WITH PROCESSED LIPOASPIRATE CELLS, and U.S. application Ser. No. 10/877,822 (U.S. Pub. No. 2005/0084961) entitled SYSTEMS AND METHODS FOR SEPARATING AND CONCENTRATING REGENERATIVE CELLS FROM TISSUE, the contents of each incorporated herein by reference. The regenerative cells may be manipulated by chemical agents or additives, biological agents or additives, or physical stimuli.

The system for separating and concentrating adipose-derived regenerative cells can comprise tests for confirming that the appropriate level of manipulation has occurred. These tests can be based upon input by the user whereby the system automatically selects (e.g., from a stored set of tests that the system is capable of accommodating, facilitating or at least partially performing) a group of tests. The system then automatically displays (or otherwise conveys to the user) these tests and, optionally, prompts the user to choose from among the displayed tests.

In another embodiment, the system is automated such that the entire method, including separation, concentration, and manipulation of the regenerative cells, may be performed in a continuous sequence with minimal user intervention. In embodiments, the entire procedure from tissue extraction through separating, concentrating, manipulation and placement of the regenerative cells into the recipient are performed in the same facility, indeed, even within the same room of the patient undergoing the procedure. The regenerative cells may be used in a relatively short time period after separation, concentration and manipulation. For example, the regenerative cells may be ready for use in about one to two hours from the harvesting of tissue from a patient, and in certain situations, may be ready for use in about 10 to 40 minutes from the harvesting of the tissue. The entire length of the procedure from extraction through separation, concentration and manipulation may vary depending on a number of factors, including patient profile, type of tissue being harvested and the amount or type of manipulation of the regenerative cells required for a given therapeutic application. For example, the manipulation may require a few days. The cells can also be placed into the recipient in combination with other cells, tissue, tissue fragments, scaffolds or other stimulators of cell growth and/or differentiation in the context of a single operative procedure with the intention of providing a therapeutic, structural, or cosmetic benefit to the recipient. For example, the reinfusion into a patient may be via placement onto an osteoconductive scaffold for orthopaedic applications.

Patients suffering from a wide variety of diseases and disorders may benefit from the regenerative cells of the present invention. For example, patients suffering from cardiovascular diseases and disorders, liver diseases and disorders, renal diseases and disorders, skeletal muscle disorders, lung injuries and disorders, diabetes, intestinal diseases and disorders, nervous system disorders, Parkinson's disease, Alzheimer's, stroke related diseases and disorders, diseases and disorders of the hematopoietic system, wounds, ulcers and other diseases and disorders of the skin, traumatic injury, burn, radiation or chemical or other toxin-induced injuries or disorders, and bone and cartilage-related diseases and disorders can be treated using the regenerative cells obtained through the systems and methods of the present invention.

In particular embodiments, diseases and disorders that are mediated by angiogenesis, arteriogenesis, or the inflammatory response can be treated with the regenerative cells obtained using the systems and methods of the present invention. For example, acute myocardial infarctions, ischemic cardiomyopathy, peripheral vascular disease, ischemic stroke, acute tubular necrosis, ischemic wounds, sepsis, ischemic bowel disease, diabetic retinopathy, neuropathy, nephropathy, vasculitidies, ischemic encephalopathy, erectile dysfunction, ischemic and/or traumatic spinal cord injuries, multiple organ system failures, ischemic gum disease and transplant related ischemia can be treated.

Furthermore, diseases and disorders affecting more than one physiological system, e.g., traumatic injury involving both soft and hard tissues, the effects of aging, multi-organ disorders, etc., may also be treated with the regenerative cells obtained using the systems and methods of the present invention. The regenerative cells can also be used to promote tendon and cartilage repair and for a variety of clinical and non-clinical cosmetic and structural applications, including autologous fat transfer applications. Cosmetic applications include, for example, restructuring of facial folds and wrinkles, lip, breast and buttocks as well as other soft tissue defects (e.g., caused by radionecrosis, e.g., following radiation or chemotherapy for treatment of breast or other cancers). The regenerative cells may also be used for tissue engineering applications known in the art.

### DEFINITIONS

In order that the present invention may be more readily understood, certain terms are first defined. Additional definitions are set forth throughout the detailed description. Unless otherwise indicated, all terms used herein have the same ordinary meaning as they would to one skilled in the art of the present invention.

As used herein, “regenerative cells” refers to any heterogeneous or homologous cells obtained using the systems and methods of the present invention which cause or contribute to complete or partial regeneration, restoration, or substitution of structure or function of an organ, tissue, or physiologic unit or system to thereby provide a therapeutic, structural or cosmetic benefit. Examples of regenerative cells include: ASCs, endothelial cells, endothelial precursor cells, endothelial progenitor cells, macrophages, fibroblasts, pericytes, smooth muscle cells, preadipocytes, differentiated or de-differentiated adipocytes, keratinocytes, unipotent and multipotent progenitor and precursor cells (and their progeny), and lymphocytes.

One mechanism by which the regenerative cells may provide a therapeutic, structural or cosmetic benefit is by incorporating themselves or their progeny into newly generated, existing or repaired tissues or tissue components. For example, ASCs and/or their progeny may incorporate into newly generated bone, muscle, or other structural or functional tissue and thereby cause or contribute to a therapeutic, structural or cosmetic improvement. Similarly, endothelial cells or endothelial precursor or progenitor cells and their progeny may incorporate into existing, newly generated, repaired, or expanded blood vessels to thereby cause or contribute to a therapeutic, structural or cosmetic benefit.

Another mechanism by which the regenerative cells may provide a therapeutic, structural or cosmetic benefit is by expressing and/or secreting molecules, e.g., growth factors, that promote creation, retention, restoration, and/or regeneration of structure or function of a given tissue or tissue component. For example, regenerative cells may express and/or secrete molecules which result in enhanced growth of tissues or cells that then participate directly or indirectly in improved structure or function. Regenerative cells may express and/or secrete growth factors, including, for example, Vascular Endothelial Growth Factor (VEGF), Placental Growth factor (PlGF), bFGF, IGF-II, Eotaxin, G-CSF, GM-CSF, IL-12 p40/p70, IL-12 p70, IL-13, IL-6, IL-9, Leptin, MCP-1, M-CSF, MIG, PF-4, TIMP-1, TIMP-2, TNF-α, Thrombopoetin, and their isoforms, which may perform one or more of the following functions: stimulate development of new blood vessels, i.e., promote angiogenesis; improve oxygen supply of pre-existent small blood vessels (collaterals) by expanding their blood carrying capacity; induce mobilization of regenerative cells from sites distant from the site of injury to thereby enhance the homing and migration of such cells to the site of injury; stimulate the growth and/or promote the survival of cells within a site of injury thereby promoting retention of function or structure; deliver molecules with anti-apoptotic properties thereby reducing the rate or likelihood of cell death and permanent loss of function; and interact with endogenous regenerative cells and/or other physiological mechanisms.

The regenerative cells may be used in their ‘native’ form as present in or separated and concentrated from the tissue using the systems and methods of the present invention or they may be modified by stimulation or priming with growth factors or other biologic response modifiers, by gene transfer (transient or stable transfer), by further sub-fractionation of the resultant population on the basis or physical properties (for example size or density), differential adherence to a solid phase material, expression of cell surface or intracellular molecules, cell culture or other ex vivo or in vivo manipulation, modification, or fractionation as further described herein. The regenerative cells may also be used in combination with other cells or devices such as synthetic or biologic scaffolds, materials or devices that deliver factors, drugs, chemicals or other agents that modify or enhance the relevant characteristics of the cells as further described herein.

As used herein, “regenerative cell composition” refers to the composition of cells typically present in a volume of liquid after a tissue, e.g., adipose tissue, is washed and at least partially disaggregated. For example, a regenerative cell composition of the invention comprises multiple different types of regenerative cells, including ASCs, endothelial cells, endothelial precursor cells, endothelial progenitor cells, macrophages, fibroblasts, pericytes, smooth muscle cells, preadipocytes, differentiated or de-differentiated adipocytes, keratinocytes, unipotent and multipotent progenitor and precursor cells (and their progeny), and lymphocytes. The regenerative cell composition may also contain one or more contaminants, such as collagen, which may be present in the tissue fragments, or residual collagenase or other enzyme or agent employed in or resulting from the tissue disaggregation process described herein.

As used herein, “regenerative medicine” refers to any therapeutic, structural or cosmetic benefit that is derived from the placement, either directly or indirectly, of regenerative cells into a subject. Regenerative medicine encompasses all of the diseases and disorders described herein as well as those known in the art.

As used herein, “stem cell” refers to a multipotent regenerative cell with the potential to differentiate into a variety of other cell types, which perform one or more specific functions and have the ability to self-renew. Some of the stem cells disclosed herein may be multipotent.

As used herein, “progenitor cell” refers to a multipotent regenerative cell with the potential to differentiate into more than one cell type and has limited or no ability to self-renew. “Progenitor cell,” as used herein, also refers to a unipotent cell with the potential to differentiate into only a single cell type, which performs one or more specific functions and has limited or no ability to self-renew. In particular, as used herein, “endothelial progenitor cell” refers to a multipotent or unipotent cell with the potential to differentiate into vascular endothelial cells.

As used herein, “precursor cell” refers to a unipotent regenerative cell with the potential to differentiate into one cell type. Precursor cells and their progeny may retain extensive proliferative capacity, e.g., lymphocytes and endothelial cells, which can proliferate under appropriate conditions.

As used herein “stem cell number” or “stem cell frequency” refers to the number of colonies observed in a clonogenic assay in which adipose derived cells (ADC) are plated at low cell density (<10,000 cells/well) and grown in growth medium supporting MSC growth (for example, DMEM/F12 medium supplemented with 10% fetal calf serum, 5% horse serum, and antibiotic/antimycotic agents). Cells are grown for two weeks after which cultures are stained with hematoxylin and colonies of more than 50 cells are counted as CFU-F. Stem cell frequency is calculated as the number of CFU-F observed per 100 nucleated cells plated (for example; 15 colonies counted in a plate initiated with 1,000 nucleated regenerative cells gives a stem cell frequency of 1.5%). Stem cell number is calculated as stem cell frequency multiplied by the total number of nucleated ADC cells obtained. A high percentage (˜100%) of CFU-F grown from regenerative cells express the cell surface molecule CD105 which is also expressed by marrow-derived stem cells (Barry et al., 1999, Biochem Biophys Res Commun 265(1):134-9). CD105 is also expressed by adipose tissue-derived stem cells (Zuk et al., 2002, Mol Biol Cell 13(12):4279-95).

As used herein, the term “adipose tissue” refers to fat including the connective tissue that stores fat. Adipose tissue contains multiple regenerative cell types, including ASCs and endothelial progenitor and precursor cells.

As used herein, the term “unit of adipose tissue” refers to a discrete or measurable amount of adipose tissue. A unit of adipose tissue may be measured by determining the weight and/or volume of the unit. Based on the data identified above, a unit of processed lipoaspirate, as removed from a patient, has a cellular component in which at least 0.1% of the cellular component is stem cells; that is, it has a stem cell frequency, determined as described above, of at least 0.1%. In reference to the disclosure herein, a unit of adipose tissue may refer to the entire amount of adipose tissue removed from a patient, or an amount that is less than the entire amount of adipose tissue removed from a patient. Thus, a unit of adipose tissue may be combined with another unit of adipose tissue to form a unit of adipose tissue that has a weight or volume that is the sum of the individual units.

As used herein, the term “portion” refers to an amount of a material that is less than a whole. A minor portion refers to an amount that is less than 50%, and a major portion refers to an amount greater than 50%. Thus, a unit of adipose tissue that is less than the entire amount of adipose tissue removed from a patient is a portion of the removed adipose tissue.

As used herein, the term “processed lipoaspirate” refers to adipose tissue that has been processed to separate the active cellular component (e.g., the component containing regenerative cells) from the mature adipocytes and connective tissue. This fraction is referred to herein as “adipose-derived cells” or “ADC.” Typically, ADC refers to the pellet of regenerative cells obtained by washing and separating and concentrating the cells from the adipose tissue. The pellet is typically obtained by centrifuging a suspension of cells so that the cells aggregate at the bottom of a centrifuge chamber or cell concentrator.

As used herein, the terms “administering,” “introducing,” “delivering,” “placement” and “transplanting” are used interchangeably herein and refer to the placement of the regenerative cells of the invention into a subject by a method or route which results in at least partial localization of the regenerative cells at a desired site. The regenerative cells can be administered by any appropriate route which results in delivery to a desired location in the subject where at least a portion of the cells or components of the cells remain viable. The period of viability of the cells after administration to a subject can be as short as a few hours, e.g., twenty-four hours, to a few days, to as long as several years.

As used herein, the term “treating” includes reducing or alleviating at least one adverse effect or symptom of a disease or disorder.

As used herein, the term “delivery” is intended to encompass all means particularly described herein and known in the art by which cells are placed within a recipient. Such means include, but are not limited to, injection into intravascular, intramuscular, periurethral, subcutaneous tissues and spaces, injectable solutions (e.g., injectable solutions using hyaluronon or hyaluronic acid), implantation in combination with a scaffold or carrier, e.g., resorbable scaffolds, beads, microspheres, nanospheres, hydrogels, gels, polymers, ceramics, collagen, and platelet gels, hyaluronon based scaffolds or gels, apatite-coated scaffolds or gels, PLA-based scaffolds, self-assembled peptides or any combinations thereof, implantation in combination with other cells or tissue or tissue fragments (e.g., in combination with adipocytes as in for soft tissue applications), timed release (e.g., through drug-eluting stents), etc.

As used herein, “therapeutically effective dose of regenerative cells” refers to an amount of regenerative cells that is sufficient to bring about a beneficial or desired clinical effect. Said dose could be administered in one or more administrations. However, the precise determination of what would be considered an effective dose may be based on factors individual to each patient, including, but not limited to, the patient's age, size, type or extent of disease, stage of the disease, route of administration of the regenerative cells, the type or extent of supplemental therapy used, ongoing disease process and type of treatment desired (e.g., aggressive vs. conventional treatment).

As used herein, the term “subject” includes warm-blooded animals, preferably mammals, including humans. In a preferred embodiment, the subject is a primate. In an even more preferred embodiment, the subject is a human.

### MODES OF CARRYING OUT THE INVENTION

It is to be understood that this invention is not limited to particular formulations or process parameters, as these may, of course, vary. It is also to be understood that the terminology used herein is for the purpose of describing particular embodiments of the invention only, and is not intended to be limiting. Further, it is understood that a number of methods and materials similar or equivalent to those described herein can be used in the practice of the present invention.

I. Harvesting Regenerative Cells from Adipose Tissue

As previously discussed, regenerative cells, e.g., stem and progenitor cells, can be harvested from a wide variety of tissues. The systems described herein or incorporated by reference may be used for all such tissues. Adipose tissue, however, is an especially rich source of regenerative cells. Accordingly, the methods of manipulation of the present invention are described herein using adipose tissue as a source of regenerative cells by way of example only and not limitation.

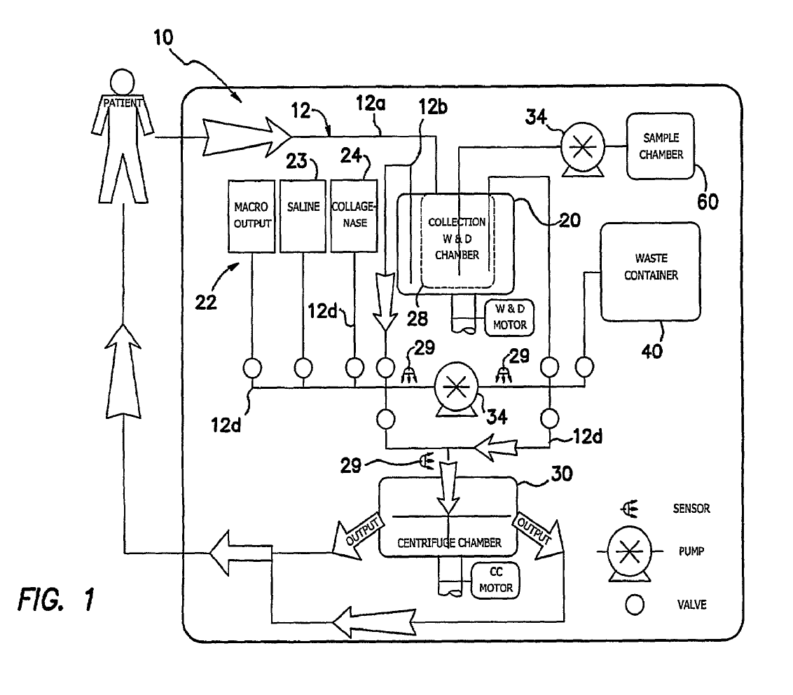

An exemplary system for harvesting, shown schematically in FIG. 1 and described in U.S. application Ser. No. 10/884,638, incorporated herein by reference, is generally comprised of one or more of a tissue collection chamber 20, a processing chamber 30, a waste chamber 40, an output chamber 50 and a sample chamber 60. The various chambers are coupled together via one or more conduits 12 such that fluids containing biological material may pass from one chamber to another while maintaining a closed or functionally closed, sterile fluid/tissue pathway. A functionally closed pathway refers to a system, of an otherwise structurally closed system of bags, tubing, and other components, that is penetrated solely in an aseptic or sterile fashion. Typically, this includes addition of materials through a sealed rubber septum that has been cleaned by wiping with alcohol, povidone iodine or similar agent, through a luer-lock-type fitting in an aseptic or sterile environment, or through a temporary opening that, while open, is maintained within an aseptic or sterile environment. In one embodiment, such a system includes means of chemically and/or physically manipulating the regenerative cells. In another embodiment, such a system is attached via the use of sterile connecting devices to a second closed system in a closed, sterile or aseptic fashion. In this embodiment, the second closed system includes means of chemically and/or physically manipulating the regenerative cells. In certain embodiments, both the first and the second closed systems include means of chemically, biologically and/or physically manipulating the regenerative cells.

For most applications preparation of the active cell population will require depletion of the mature fat-laden adipocyte component of adipose tissue. This is typically achieved by a series of washing and disaggregation steps in which the tissue is first rinsed, for example, in the tissue collection chamber 20, to reduce the presence of free lipids (released from ruptured adipocytes) and peripheral blood elements (released from blood vessels severed during tissue harvest), and then disaggregated to free intact adipocytes and other cell populations from the connective tissue matrix. In certain embodiments, the entire adipocyte component or a portion of the adipocyte component is separated from other components of the adipose tissue.

Rinsing is an optional step in which the tissue is mixed with solutions to wash off free lipid and single cell components, such as those components in blood, leaving behind intact adipose tissue fragments. In one embodiment, the adipose tissue that is removed from the patient is mixed with isotonic saline or other physiologic solution(s) (e.g., Plasmalyte® of Baxter Inc., Normosol® of Abbott Labs, or Lactated Ringers' Solution). Intact adipose tissue fragments can be separated from the free lipid and cells by any means known to persons of ordinary skill in the art including, but not limited to, filtration, decantation, sedimentation, or centrifugation. In embodiments of the invention, the adipose tissue is separated from non-adipose tissue by employing a filter disposed within a tissue collection container, as discussed herein. In other embodiments, the adipose tissue is separated from non-adipose tissue using a tissue collection container that utilizes decantation, sedimentation, and/or centrifugation techniques to separate the materials.

The intact tissue fragments are then disaggregated using any conventional techniques or methods, including mechanical force (mincing or shear forces), enzymatic digestion with single or combinatorial proteolytic enzymes, such as collagenase, trypsin, lipase, liberase H1, or members of the Blendzyme family as disclosed in U.S. Pat. No. 5,952,215, expressly incorporated herein by reference in its entirety, and pepsin, or a combination of mechanical and enzymatic methods. For example, the cellular component of the intact tissue fragments may be disaggregated by methods using collagenase-mediated dissociation of adipose tissue, similar to the methods disclosed in U.S. Pat. No. 5,372,945, expressly incorporated herein by reference in its entirety. Additional methods using collagenase are disclosed in U.S. Pat. Nos. 5,830,714 and 5,952,215, and by Williams, et al. (Williams, et al., 1995, Cell Transplant. 4(3):281-9), all expressly incorporated herein by reference in their entirety. Similarly, a neutral protease may be used instead of collagenase, as disclosed in Twentyman, et al. (Twentyman, et al., 1980, Cancer Lett. 9(3):225-8), expressly incorporated herein by reference in its entirety. Furthermore, methods may employ a combination of enzymes, such as a combination of collagenase and trypsin or a combination of an enzyme, such as trypsin, and mechanical dissociation. Disaggregation can also be performed in the tissue collection chamber.

Adipose tissue-derived cells may then be obtained from the disaggregated tissue fragments by reducing the presence of mature adipocytes. A suspension of the disaggregated adipose tissue and the liquid in which the adipose tissue was disaggregated is then passed to another container, such as a cell collection container or the processing chamber 30. The suspension may flow through one or more conduits to the cell collection container or processing chamber by using a pump, such as a peristaltic pump, that withdraws the suspension from the tissue collection container and urges it to the cell collection container. Other embodiments may employ the use of gravity or a vacuum while maintaining a closed system. Separation of the cells in the suspension may be achieved by buoyant density sedimentation, centrifugation, elutriation, filtration, differential adherence to and elution from solid phase moieties, antibody-mediated selection, differences in electrical charge; immunomagnetic beads, fluorescence activated cell sorting (FACS), or other means. Examples of these various techniques and devices for performing the techniques may be found in U.S. Pat. Nos. 6,277,060; 6,221,315; 6,043,066; 6,451,207; 5,641,622; and 6,251,295, all incorporated herein by reference in their entirety.

In embodiments, the cells in the suspension are separated from the acellular component of the suspension using a spinning membrane filter. In other embodiments, the cells in the suspension are separated from the acellular component using a centrifuge. In one such exemplary embodiment, the cell collection container may be a flexible bag that is structured to be placed in a centrifuge (e.g., manually or by robotics). In other embodiments, a flexible bag is not used. Centrifugation can take place within the processing chamber 30 or in a separate chamber. After centrifugation, the cellular component forms a pellet, which may then be resuspended with a buffered solution so that the cells can be passed through one or more conduits to a mixing container, as discussed herein. The resuspension fluids may be provided by any suitable means. For example, a buffer may be injected into a port on the cell collection container, or the cell collection container may include a reserve of buffer that can be mixed with the pellet of cells by rupturing the reserve. When a spinning membrane filter is used, resuspension is optional since the cells remain in a volume of liquid after the separation procedure.

Although certain embodiments of the invention are directed to methods of fully disaggregating the adipose tissue to separate the active cells from the mature adipocytes and connective tissue, additional embodiments of the invention are directed to methods in which the adipose tissue is only partially disaggregated. For example, partial disaggregation may be performed with one or more enzymes, which are removed from at least a part of the adipose tissue early relative to an amount of time that the enzyme would otherwise be left thereon to fully disaggregate the tissue.

Following disaggregation the active cell population can be washed/rinsed to remove additives and/or by-products of the disaggregation process (e.g., collagenase and newly-released free lipid). The active cell population can then be concentrated by centrifugation or other methods known to persons of ordinary skill in the art, as discussed above. These post-processing wash/concentration steps may be applied separately or simultaneously.

In one embodiment, the cells are concentrated and the collagenase removed by passing the cell population through a continuous flow spinning membrane system or the like, such as, for example, the system disclosed in U.S. Pat. Nos. 5,034,135 and 5,234,608.

In addition to the foregoing, there are many post-wash methods that may be applied for further purifying the active cell population. These include both positive selection (selecting the target cells), negative selection (selective removal of unwanted cells), or combinations thereof.

Alternatively a separate tissue collecting container, such as that described in patent application Ser. No. 10/242,094, entitled PRESERVATION OF NON EMBRYONIC CELLS FROM NON HEMATOPOIETIC TISSUES, filed Sep. 12, 2002, which claims the benefit of U.S. App. Ser. No. 60/322,070 filed Sep. 14, 2001, both incorporated herein by reference, could be employed in whole or in part with subsequent transfer of the disaggregated material to the processing components. Additional potential tissue collecting containers are disclosed in U.S. Pat. Nos. 6,316,247 and 5,372,945, both of which are expressly incorporated herein by reference.

Many other conformations of the staged mechanisms used for cell processing will be apparent to one skilled in the art. The present description is included as one example only. For example, mixing of tissue and saline during washing and disaggregation can occur by agitation as in the present example or by fluid recirculation. Cell washing may be mediated by a continuous flow mechanism such as the spinning membrane approach, differential adherence, differential centrifugation (including, but not limited to differential sedimentation, velocity, or gradient separation), or by a combination of means. Similarly, additional components allow further manipulation of cells, including addition of growth factors or other biological response modifiers, and mixing of cells with natural or synthetic components intended for implant with the cells into the recipient.

Post-processing manipulation may also include cell culture or further cell purification (Kriehuber, et al., 2001, J. Exp. Med. 194(6): 797-808; Garrafa, et al., 2006, J. Cell Physiol. 207(1): 107-13). Mechanisms for performing these functions may be integrated within the described device or may be incorporated in separate devices.

II. Methods of Manipulating Regenerative Cells from Adipose Tissue

Manipulation of the regenerative cell sample using the methods of the present invention can be performed in order to, e.g., separate sample components, enrich for desired components and/or eliminate undesired components, enhance the beneficial effect of treatment with the regenerative cells, or decrease undesired effects. In embodiments, enhancing certain functions of the regenerative cells or increasing the cells' viability are contemplated. The invention also contemplates manipulations that perform multiple functions, and subjecting the regenerative cell sample to multiple manipulations before administration.

For chemical or biological manipulation, manipulating agents may be introduced via any of the ports, conduits, tubings, lumens, valves or pumps. The chemical or biological additives or agents may also be provided with any of the ports, conduits, tubings, lumens, valves, pumps, filters or chambers, including the disposable sets. For example, the chemical or biological agents or additives may be coated onto any of the chambers, disposable sets or other components of the device. The chemical or biological additives or agents may also be stored in specific ports of the system and may be released during various stages of the processing method or may be released in a time-released and/or continuous fashion. These chemical or biological additives or agents may also be provided as part of another system associated with the existing system or separate from the existing system. For example, in certain embodiments, the additives are added or provided without the need for removing the regenerative cells from the system. In other embodiments, the additives are added or provided by connecting a new container or chamber comprising the additives into an unused port of the system in a sterile manner. In yet other embodiments, the additives are added or provided in a second system or device that is not connected to the system of the present invention. It is contemplated that additives or agents are provided at any time, e.g., before the sample is added to the machine, at any time during processing within the machine, or after completion of processing.

### A. Additives

For example, during washing and/or disaggregation, one or more additives may be added to the various containers as needed to enhance the results. Some examples of additives include agents that optimize washing and disaggregation, additives that enhance the viability of the active cell population during processing, anti-microbial agents (e.g., antibiotics), additives that lyse adipocytes and/or red blood cells, or additives that enrich for cell populations of interest (by differential adherence to solid phase moieties or to otherwise promote the substantial reduction or enrichment of cell populations, e.g., by selective attachment). Other possible additives include those that promote recovery and viability of regenerative cells (for example, caspase inhibitors) or which reduce the likelihood of adverse reaction on infusion or placement (for example, inhibitors of re-aggregation or clumping of cells or connective tissue). Also contemplated are modulators of apoptosis and inflammation, many of which are known in the art and have been described in the literature. In embodiments, all of the additives are regenerative cell friendly, i.e., are not toxic or are minimally toxic to the regenerative cells or the intended recipient. The additives described herein preferably do not cause the regenerative cells to adhere or stick to the additives and thereby to any of the containers, chambers, disposable sets, ports, valves, conduits, tubings, etc. In yet other embodiments, particular additives are used such that they actively prevent the regenerative cells from adhering or sticking to any of the components of the system with which the regenerative cells are in contact (e.g., to optimize regenerative cell yield).

Effective concentrations of additives can be determined using methods described herein and known to those of skill in the art.

### (i) Disaggregation Agents

An additive comprising a tissue disaggregation agent can be delivered, e.g., to separate the regenerative cells from the remaining adipose tissue components. The disaggregation agent can be any disaggregation agent known to one of skill in the art. Digestive enzymes for use in cell isolation from tissue are described, e.g., in U.S. Pat. Pub. No. 2005/0058630, incorporated herein by reference in its entirety. Contemplated enzymes range from those considered weakly digestive (e.g. deoxyribonucleases and the neutral protease, dispase) to strongly digestive (e.g. papain and trypsin). For example, collagenases are known to be useful for isolating various cells from tissues. Multiple enzymes can be used in combination or sequentially.

In a specific embodiment, the agent is added directly to the collection chamber 20 where it is mixed with the washed tissue sample and where digestion is allowed to take place. The agent can also be added to the processing chamber 30 or other chambers. Specific examples of disaggregation agents that can be used include neutral proteases, collagenase, trypsin, lipase, hyaluronidase, nucleases, e.g., deoxyribonucleases, members of the Blendzyme enzyme mixture family, e.g., Liberase H1, pepsin, ultrasonic or other physical energy, lasers, microwaves, other mechanical devices and/or combinations thereof. A contemplated disaggregation agent is collagenase. The disaggregation agents can be added with other solutions and in combination or in sequence with one or more other disaggregation agents. For example, saline, such as saline delivered from a saline source 23 as shown in FIG. 1, may be added to the adipose tissue along with or immediately followed by addition of collagenase. In one embodiment, the washed adipose tissue is mixed with a collagenase-containing enzyme solution at or around 37° C. for about 20-60 minutes. In other embodiments, a higher concentration of collagenase or similar agent may be added to decrease the digestion time. The washed adipose tissue and the tissue disaggregation agent can then be agitated using any of the agitation methods described above, or similar methods, until the washed adipose tissue is disaggregated to the extent desired. For example, the washed adipose tissue and the tissue disaggregation agent may be agitated by rotating the entire collection chamber through an arc of approximately 90 degrees, by having a shaft containing one or more paddles which pass through the solution as the shaft is being rotated, and/or by rotating the entire collection chamber which contains paddles or protrusions on the inside surface of the collection chamber.

In embodiments, the tissue is washed, with sterile buffered isotonic saline or Lactated Ringers Solution, and incubated with collagenase at a collagenase concentration, a temperature, and for a period of time sufficient to provide adequate disaggregation. In embodiments, the collagenase enzyme used will be approved for human use by the relevant authority (e.g., the U.S. Food and Drug Administration). Suitable collagenase preparations include recombinant and non-recombinant collagenase. Non-recombinant collagenase may be obtained from F. Hoffmann-La Roche Ltd., Indianapolis, Ind. and/or Advance Biofactures Corp., Lynbrook, N.Y. Recombinant collagenase may also be obtained as disclosed in U.S. Pat. No. 6,475,764.

Depending on the purpose for which the adipose derived cells will be used, the adipose tissue may either be partially disaggregated, or completely disaggregated. The degree of disaggregation can be determined using any of a number methods, e.g., by measuring current flow through, optical density of, or color change in the sample or the waste solution at different times following addition of the disaggregating agent. A measuring system could be based, for example, on a correlation between the degree of disaggregation determined by evaluation of the sample, e.g., using a hemocytometer, and a selected value obtained by testing one or more parameters, e.g., the electrical impedance of the sample in the container. In embodiments, upon detection of insufficient disaggregation, the disaggregation step is allowed to continue and the degree of disaggregation tested again.

Furthermore, deoxyribonucleases can digest single-stranded DNA and can minimize cell clumping during isolation. In embodiments, an appropriate deoxyribonuclease, e.g., DNAse I, is added at a concentration determined to effectively reduce cell clumping using procedures known to those of skill in the art. For example, human DNAse I can be added to the washed, disaggregated tissue in a processing chamber, e.g., the collection chamber 20 or centrifuge chamber 30 (FIG. 1) and digestion allowed to take place in saline or Lactated Ringers solution or other effective solution at an appropriate temperature, e.g., at room temperature. Nuclease digestion is allowed continue for a period of time, e.g., 10 minutes, 20 minutes, or until a time when clumping has been eliminated to a level of satisfaction, as determined by methods known in the art. Following digestion, residual DNAse can be removed using a washing procedure known in the art, e.g. by centrifugation and subsequent resuspension of the cells in saline or Lactated Ringers solution.

### (ii) Other Additives

Other examples of additives may include additional biological or structural components, such as cell differentiation factors, cell de-differentiation factors, growth promoters, immunosuppressive agents, anti-apoptotic agents, anti-inflammatory agents, medical devices, or any combinations thereof, as discussed herein. For example, other cells, tissue, tissue fragments, growth factors such as VEGF and other known angiogenic or arteriogenic growth factors, biologically active or inert compounds, e.g., cardiogenol C or creatine, resorbable scaffolds, or other additives intended to enhance the delivery, efficacy, tolerability, or function of the population of regenerative cells may be added. For example, the cells could be given a priming boost, for example, using HGF which can cause increased sensitivity to chemoattractants.

When the cells and/or tissue containing the cells are administered to a patient other than the patient from whom the cells and/or tissue were obtained, one or more immunosuppressive agents may be administered to the patient receiving the cells and/or tissue to reduce, and preferably prevent, rejection of the transplant. As used herein, the term “immunosuppressive drug or agent” is intended to include pharmaceutical agents which inhibit or interfere with normal immune function. Examples of immunosuppressive agents suitable with the methods disclosed herein include agents that inhibit T-cell/B-cell costimulation pathways, such as agents that interfere with the coupling of T-cells and B-cells via the CTLA4 and B B7 pathways, as disclosed in U.S. Patent Pub. No. 2002/0182211. A contemplated immunosuppressive agent is cyclosporine A. Other examples include myophenylate mofetil, rapamicin, and anti-thymocyte globulin. In one embodiment, the immunosuppressive drug is administered with at least one other therapeutic agent. The immunosuppressive drug is administered in a formulation which is compatible with the route of administration and is administered to a subject at a dosage sufficient to achieve the desired therapeutic effect. In another embodiment, the immunosuppressive drug is administered transiently for a sufficient time to induce tolerance to the regenerative cells of the invention. A suitable immunosuppressive drug or agent could be housed, e.g., in a special port of the system and released and incorporated into the regenerative cell pellet if prompted by the user or automatically based on the program selected by the user.

In any of the embodiments described herein, the additives may be contacted, combined, mixed or added to the regenerative cells through any art recognized manner, including devices such as the agitation devices and associated methods described herein. For example, rocking, inversion, compression pulsed or moving rollers may be used.

### B. Modification of Regenerative Cells

The regenerative cell population may also be modified by insertion of DNA or by placement in a cell culture system, as described herein or known in the art, in such a way as to change, enhance, or supplement the function of the regenerative cells for derivation of a structural or therapeutic purpose. For example, gene transfer techniques for stem cells are known by persons of ordinary skill in the art, as disclosed in Morizono et al., 2003, Hum. Gene Ther. 14(1):59-66 and Mosca et al., 2000, Clin. Orthop. Supp 379:71-90, and may include viral transfection techniques, more specifically, adeno-associated virus gene transfer techniques, as disclosed by Walther et al., 2000, Drugs 60(2):249-71 and Athanasopoulos et al., 2000, Int. J. Mol. Med. 6(4):363-75. Non-viral based techniques may also be performed as disclosed in Muramatsu et al., 1998, Int. J. Mol. Med. (1):55-62. A gene encoding one or more cellular differentiating factors, e.g., a growth factor(s) or a cytokine(s), could also be added. Examples of various cell differentiation agents are reported by Gimble et al., 1995, J. Cell Biochem. 58(3):393-402; Lennon et al., 1995, Exp Cell Res 219(1):211-22; Majumdar et al., 1998, J. Cell Physiol. 176(1): 57-66; Caplan and Goldberg, 1999, Clin Orthop Suppl. 367: 12-16; Ohgushi and Caplan, 1999, J. Biomed. Mater. Res. 48(6):913-27; Pittenger et al., 1999, Science 284(5411):143-7; Caplan and Bruder, 2001, Trends Mol. Med. 7(6):259-64; Fukuda, 2001, Artif. Organs 25(3):187-93; Worster et al., 2001, J. Orthop. Res. 19(4):738-49, and; Zuk et al., 2001, Tissue Eng 7(2): 211-28. Genes encoding anti-apoptotic factors or agents can also be added.

Addition of the gene or combination of genes can be made using any technology known in the art including but not limited to adenoviral transduction, including using “gene guns,” liposome-mediated transduction, retrovirus or lentivirus-mediated transduction, plasmid, and adeno-associated virus. These regenerative cells could then be implanted along with a carrier material bearing gene delivery vehicle capable of releasing and/or presenting genes to the cells over time such that transduction can continue or be initiated in situ. These regenerative cells could also be implanted via seeding on a scaffold, e.g., a resorbable scaffold or a hydrogel, or other carrier as described herein or known in the art.

Electroporation induces transient pore formation in the plasma membrane, allowing oligonucleotides or other constructs to migrate into the cytoplasm and nucleus. This method can be used to introduce a gene that will result in killing of unwanted cells. For example, it was reported that a plasmid vector in which an antibiotic (Zeocin) resistance gene driven by MyoD and Myf5 enhancer elements, selectively active in skeletal muscle progenitor cells, was introduced into MSCs obtained from mouse bone marrow, allowing myogenic precursor cells to be isolated by antibiotic selection (Bhagavati, et al., 2004, Biochem. Biophys. Res. Comm. 318(1):318-24.)

Reportedly, synthetic antisense oligonucleotides have been introduced into bone marrow cells to achieve selective removal of cells (Bergan, et al., 1996, Blood 88(2):731-741). Furthermore, FITC-dextran macromolecule electroloading of ADCs has been successfully demonstrated, and electroporation of ADCs with pCMV-GFP plasmid resulted in >70% of processed cells expressing GFP at day 2 post-transfection (data not shown).

In embodiments, modification of the cells with genes encoding agents is made after sample washing and disaggregation, and can be made after at least one separation step to enhance for a desired cell type or types. In other embodiments, the modified or unmodified cells are seeded on or otherwise combined with a scaffold just prior to administration to the patient.

### C. Physical Manipulation of Cells

Physical manipulation can influence one or more aspects of the regenerative cells' behavior. Certain physical manipulations may be accomplished via, for example, one or more temperature control devices, agitation devices, rotation devices or other physical mechanisms existing in the system described herein, incorporated by reference or known in the art. Alternatively, additional devices, light sources, heat sources or chambers that perform specific physical manipulation of the regenerative cells may be added.

### (i) Hypoxia and Hyperoxia

A low oxygen environment can affect cell metabolism. Effects on differentiation of cells subjected to hypoxic conditions have been reported by, e.g., Di Carlo, et al., 2004, JBC 279(16):16332-338, Fink, et al., 2004, Stem Cells 22: 1346-1355, and Rolovic, et al., 1990, Exp. Hematol. 18(3):190-4. Elevation of markers for osteogenesis, including alkaline phosphatase activity, calcium content, and von Kossa staining, in low oxygen cultures have also been reported (Lennon, et al., 2001, J. Cell Phys. 187(3):345-55). Furthermore, specific genes, including genes encoding isoforms of Hypoxia-Inducing Factor 1 (HIF-1), and the genes encoding GLUT-1 and MMP-2, are reportedly expressed by nucleus pulposus cells, which exist in the low-oxygen environment in spinal discs (Rajpurohit, et al., 2002, Cell Tissue Res. 308(3):401-7). Rehman, et al., in Circulation 109:1292-98 (2004) reported exposing human ASCs (adipose stromal cells) to either normoxic (21% O₂) or hypoxic (1% O₂) conditions.

Hypoxia can alter gene expression by hypoxia-sensitive cells through induction of genes such as members of regulatory gene family of Hypoxia-Inducible Factors (HIFs, such as HIF-1α). For example, expression of HIF-1α gene induces expression of genes associated with non-oxidative metabolism (glycolysis and glucose transport) thereby improving the survival of cells in a hypoxic environment. Pre-exposing ADC to hypoxia may, therefore, induce these genes and prepare the cells better for implantation into a hypoxic environment such as an ischemic tissue. Hypoxia is also associated with HIF-1-induced expression of Vascular Endothelial Growth factor (VEGF) a factor shown to be expressed by ADSC. Thus, in one embodiment, pre-delivery exposure to hypoxia can be used as a means by which post-delivery expression of VEGF by ADC may be enhanced. In addition, hypoxia can promote expression of a nucleous pulposus-like phenotype.

Hyperoxia, i.e., an increase in oxygen concentration, can also modify the phenotype and function of cells. For example, hyperoxia can augment the ability of pulmonary fibroblast like cells to transition from a lipo (adipo) phenotype to a myo (muscle-like) phenotype. Hyperoxia can also enhance neuronal differentiation of ADCs, and can inhibit proliferation of cultured smooth muscle cells. Given the ability of ADSC to differentiate into neuronal, adipocytic, and myocytic-lineages, the application of hyperoxia to ADC is a means by which their post-delivery differentiation, gene expression, and function might be manipulated.

In a particular embodiment, the physical manipulation is accomplished by manipulating the oxygen concentration, e.g., by a chamber that manipulates hypoxia or hyperoxia. Changes in oxygen concentration can be mediated by any means known to the art including use of chambers with controlled addition of oxygen and/or inert gases or by use of pharmacologic agents which interact with the cellular mechanisms by which oxygen concentration is detected in such a way that the cells respond as though the oxygen concentration is different than it actually is including agents that alter the expression and/or function of HIF-1. The cells can be incubated in the chamber at any time during processing.

### (ii) Mechanical Force

In another embodiment, physical manipulation is accomplished via the application of mechanical force. For example, exposure to cyclic equiaxial strain (e.g., for one day) can result in decreased expression of smooth muscle associated proteins. In contrast, cyclic uniaxial strain can transiently increase the expression of such genes. It is also well known that mechanical loading of bone cells is a key factor in maintenance of strength and a critical reason why astronauts returning from the microgravity of space have considerably reduced bone mineral density. This has been shown to be due to mechanosensitivity of bone cells and their precursors. Thus, given the ability of ADSC to differentiate into bone and the ability of shear force and other mechanical stimuli to promote bone cell activation, the application of such forces to ADSC is a means by which their ability to promote bone formation following delivery may be enhanced. Saha, et al. reported an inhibitory effect of biaxial cyclic strain on the differentiation of human embryonic stem cells (J. Cell Phys. 206(1):126-137, 2006). Mechanical load is also likely important in engineering of tendon cells and development of appropriate strength in the engineered construct.

Thus, the direction, frequency, and amplitude of mechanical stress or shear force can impart specific differentiation cues to developing regenerative cells. The present invention is directed, in part, at the application of such forces to adipose-derived cells over a relatively brief period (less than 48 hours) at any point of the processing method utilized by the system described herein or incorporated by reference. In embodiments, the mechanical stress or shear force is incorporated into the closed, or functionally closed system described herein such that the regenerative cells are not exposed to the risk of contamination with agents, cells, or substances that would preclude the delivery of the cells to a recipient.

Mechanical forces may also be important in the formation of blood vessels by endothelial cells and their precursors. Thus, application of mechanical forces to endothelial cells and EPCs within the ADC population is a means by which their post-delivery angiogenic and arteriogenic function might be improved. Stretch forces could also be used to, for example, induce release of the anti-apoptotic factor HGF (also expressed by ADSC) by skeletal muscle progenitor cells (satellite cells).

Application of other mechanical stimuli at any point during tissue processing in an existing or additional chamber or container, such as Low Intensity Pulsed Ultrasound (LIPUS), different intensity ultrasound (30 mW/cm2 and 120 mW/cm2), or ultrasound stimulation (200 micros pulse, 1 kHz at 30 mW/cm2), can also be used and can be applied to the sample at any point after the tissue sample is removed from a patient and the cells are administered. Low-intensity ultrasound, applied at a frequency of 0.8 MHz and intensity of 200 mW/cm² for 10 minutes a day up to 4 weeks, to rabbit mesenchymal stem cells seeded into polyglycolic acid mesh, was suggested to stimulate chondrogenesis (Cui, et al., 2006, Tissue Eng. 12(1):75-82). It was also reported that treatment with low-intensity ultrasound enhances the effect of TGF-beta on differentiation of human mesenchymal stem cells into chondrocytes (Ebisawa, et al., 2004, Tissue Eng. 10(5-6):921-9).

### (iii) Electrical and Electromagnetic Fields

In another embodiment, physical manipulation of the regenerative cells may be achieved by application of an electrical or electromagnetic field. For example, an electrical field may promote differentiation, impact development or alter gene expression.

### (iv) Pressure and Gravity

In yet another embodiment, hydrostatic pressure can be used to physically manipulate the cells to, e.g., alter the differentiation of the cells. Similarly, dynamic pressure created by fluid flow has also been shown to change the properties of cells. In addition, hypergravity can alter the functional properties of the regenerative cells (Tschopp et al, 1983, Experientia 39(12):1323-9). For example, centrifugal force can be used to generate high gravity forces and thereby create a pellet of cells at high cell density. Simulated microgravity can also influence cell phenotype.

### (v) Light

Exposure to light at different wavelengths can also modify cell behavior. For example, infra-red radiation can affect pathways associated with cell death. Thus, post-delivery survival of cells can be modified by predisposing the cells away from an apoptotic fate. Similarly, infrared-A radiation can induce changes in gene expression including expression of the matrix metalloproteinase 1 gene product. Ultraviolet light (UV) exposure can also induce changes in gene expression in different cell types. For example, UV can have an effect on the balance of expression of stimulators and inhibitors of angiogenesis including VEGF.

### (vi) Temperature Variation

In another embodiment, physical manipulation may be achieved via exposure to changes in temperature. For example, exposure to changes in temperature can alter gene expression and thereby alter cell phenotype and/or function. Heat shock has long been recognized as an inducer of stress-related genes and the whole family of Heat Shock Proteins. Priming cells with exposure to a heat shock can protect them from subsequent stress, for example the stress associated with being implanted or otherwise delivered into a recipient. Similarly, exposure to cold can induce changes in gene expression and can induce a heat shock-like response in cells that are returned to 37° C. Heat shock may be applied by any means known to the art, at any point during processing, including warming the cells and medium by radiant heat, use of laser energy, etc. Hypothermia has been reported to influence survival of cells in stem cell grafts (Miyagi, et al., 2001, Cryobiology 42:190-95).

### (vii) Osmotic Pressure

In other embodiments, physical manipulation can be achieved by application of osmotic forces. Osmotic forces, while generated by chemical or pharmacologic agents, represent a physical means by which cell phenotype and/or function may be altered. For example, osmotic forces can activate heat shock-related factors by exposure to hyper- or hypo-osmotic stress. Changes in cell responsiveness and gene expression and other cells can also result in response to agents altering osmotic pressure (Kumano et al, 1997, Adv. Perit. Dial. 13:58-63; Steffgen et al, 2003, Nephrol. Dial. Transplant. 18(11):2255-61).

### (viii) Preferential Culturing

If expansion of a regenerative cell population is required for a particular application, an approach using culture conditions to preferentially expand the population while other populations are either maintained (and thereby reduced by dilution with the growing selected cells) or lost due to absence of required growth conditions could be used. Sekiya, et al., have described conditions which might be employed in this regard for bone marrow-derived stem cells (Sekiya et al., 2002, Stem Cells 20(6):530-41). This approach (with or without differential adherence to the tissue culture plastic) could be applied to a further embodiment of this invention. In this embodiment the final regenerative cell pellet is removed from the output chamber and placed into a second system providing the cell culture component. This could be in the form of a conventional laboratory tissue culture incubator or a Bioreactor-style device such as that described by Tsao, et al., U.S. Pat. No. 6,001,642, or by Armstrong, et al., U.S. Pat. No. 6,238,908. In an alternative embodiment, the cell expansion or cell culture component could be added to the existing system, e.g., into the output chamber, allowing for short-term adherence and/or cell culture of the adipose derived cell populations. This alternate embodiment would permit integration of the cell culture and/or cell expansion component to the system and remove the need for removing the cells from this system and placement within another.

### D. Separation and Concentration Methods

To obtain a defined regenerative cell population, any suitable method for separating and concentrating the particular regenerative cell type may be employed, such as the use of cell-specific antibodies that recognize and bind antigens present on, for example, stem cells or progenitor cells, e.g., endothelial precursor cells. These include both positive selection (selecting the target cells), negative selection (selective removal of unwanted cells), or combinations thereof. Intracellular markers such as enzymes may also be used in selection using molecules which fluoresce when acted upon by specific enzymes.

The adipose tissue sample from which regenerative cells are separated can contain, in addition to the regenerative cells: extracellular matrix (ECM) debris, cell-ECM aggregates, cell debris, residual additives, anticoagulants, and other cells, e.g., blood cells. Separation of any or all of these components from the regenerative cells using the methods of the present invention is contemplated. Separation of the regenerative cells into different fractions, e.g., based on differences in cell-surface marker expression, or potential to differentiate into different cell types, is also contemplated.

It is contemplated that separation methods as described can be used at any point during sample processing. For example, separation can be made after a disaggregation step, after an additional step to eliminate cell clumping, or anytime relative to another manipulation step depending upon the type of separation being performed. Separation can be used to remove cell debris following a manipulation resulting in preferential lysis of unwanted cells. Alternatively, separation methods can be used to separate subpopulations of cells. In the latter method, one or more subpopulations could be recovered, e.g., using a valve to direct one cell population to one chamber and a second population to another chamber.

### (i) Differential Adherence

A solid phase material with adhesive properties selected to allow for differential adherence and/or elution of a particular population of regenerative cells (as described herein in the examples) within the final cell pellet could be inserted, e.g., into the output chamber of the system as described above.

Selective adherence of target regenerative cells to a matrix such as plastic (e.g., tissue culture plastic) or other solid phase surfaces (e.g., plastic beads, glass beads, etc.) may allow rapid, cost-effective target regenerative cell enrichment. This method would, for example, allow for reduction of the total number of cells delivered to a recipient without reducing the dose of effective cells. For example, separated and concentrated regenerative cells could be resuspended in a buffer and pumped into a chamber containing a matrix (e.g., tissue culture plastic, plastic beads, glass beads, or similar matrices). The regenerative cells could be incubated on that matrix for an appropriate period of time, e.g., 20 minutes to 180 minutes, for example. The matrix could be rinsed several times with the buffer to remove any non-adherent cells. The matrix could then be rinsed with a different buffer which would cause the regenerative cells to detach from the matrix. The target regenerative cells could then be further washed, concentrated, and resuspended in the desired medium suitable for reinfusion into a patient.

An alternate embodiment of this differential adherence approach would include use of antibodies and/or combinations of antibodies recognizing surface molecules differentially expressed on target regenerative cells and unwanted cells. Selection on the basis of expression of specific cell surface markers (or combinations thereof) is another commonly applied technique in which antibodies are attached (directly or indirectly) to a solid phase support structure (Geiselhart et al., 1996, Nat Immun. 15(5):227-33; Formanek et al., 1998, Eur Arch. Otorhinolaryngol. 255(4):211-55; Graepler et al., 1998, J. Biochem. Biophys. Methods 36(2-3):143-55; Kobari et al., 2001, J. Hematother. Stem Cell Res. 10(2):273-81; Mohr et al., 2001, Clin. Cancer Res. 7(1):51-57).

In another aspect, the cell population could be placed into the recipient and surrounded by a resorbable plastic sheath or other materials and related components such as those manufactured by MacroPore Biosurgery, Inc. (see e.g., U.S. Pat. Nos. 6,269,716; 5,919,234; 6,673,362; 6,635,064; 6,653,146; 6,391,059; 6,343,531; 6,280,473).

### (ii) Density Gradient Centrifugation

Density gradient centrifugation, using various separation media, including, but not limited to, Sucrose, Ficoll, Percol, and LSM (Mediatech), can be used to separate cell subsets. In density gradient centrifugation, a sample is layered over (or under) a fluid material formed into a continuous or discontinuous density gradient and placed in a centrifuge for separation of cell populations on the basis of cell density. Under centrifugal force, the particles in the sample sediment through the media in separate zones according to their density. In embodiments of the present invention, density gradient separation is automated and integrated into the regenerative cell processing method.

### (iii) Continuous Flow Centrifugation

In embodiments, continuous flow approaches such as apheresis (Smith, 1997, Ther. Apher. 1(3):203-6), and elutriation (with or without counter-current) (Lasch et al., 2000, Clin. Chem. Lab. Med. 38(7):629-32; Ito and Shinomiya, 2001, J Clin Apheresis 16(4):186-191) may also be employed.

Elutriation can be used to enrich a specific subset of cells from a fluid containing many cell types. Elutriation can thus fractionate particles in the sample and selectively retain different fractions at any point during processing, for example, after the sample is washed and leaves the collection chamber 20.

Elutriation has been described in the art, e.g., in U.S. Pat. Pub. No. 2005/0250204, incorporated herein by reference in its entirety. In one form of elutriation, a cell sample is introduced into a funnel-shaped chamber located in a spinning centrifuge. A flow of liquid elutriation buffer is then introduced into the chamber containing the cell sample. As the flow rate of the liquid buffer solution is increased through the chamber (usually in a stepwise manner), the liquid sweeps smaller sized, slower-sedimenting cells toward an elutriation boundary within the chamber, while larger, faster-sedimenting cells migrate to an area of the chamber where the centrifugal force and the sedimentation (drag) forces are balanced.

U.S. Pat. Pub. No. 2006/0086675 reports a centrifugal elutriation system configured to produce an equilibrium layer for a given sample component reported therein to extend over a widespread radial distance such that the cellular components suspended within the equilibrium layer may be better separated to allow for the effective washing of components suspended in the solution as well as to allow for ease of separation of sample components during conventional centrifugation. According to this publication, the system is capable of centrifugal elutriation of a fluid having particulate components suspended therein that may be tailored for optimized elutriation, separation, and/or suspension of selected component sizes that may be suspended in the fluid such that specific components may be selectively fractionated from the fluid.

In embodiments of the present invention, a continuous flow centrifugation device similar to that described in Example 5 below is used to concentrate and/or separate the cells. This centrifugation device could be used, for example, after the cells leave the collection chamber 20.

Advantages of continuous flow centrifugation are known and described in the art. Several variables, including centrifuge speed, pump speed, and chamber size and shape, are all controllable, allowing the user to adjust these parameters to separate certain cell fractions from others. Single or multiple cell fractions can be obtained using these methods. Continuous flow centrifugation devices further allow for fine-tuning of conditions such that the minimal force necessary is placed on the cells, thereby increasing cell viability. In embodiments of the invention, a continuous flow centrifugation device such as that described in Example 5 is used wherein the mechanical port septum is replaced with chambers having configurations that provide, e.g., higher cell recovery, greater control over cell separation, or result in less centrifugation time or higher cell viability. In all centrifugation techniques, alteration of centrifuge speed can have a significant influence on yield, viability, function, and other cell parameters.

### (iv) Dielectrophoresis Separation

Embodiments of the present invention contemplate manipulations comprising methods of separating matter derived from disaggregation of adipose tissue using dielectrophoresis and field flow fractionation. In embodiments, the manipulated matter contains regenerative cells that are suitable for placement into a recipient.

Dielectropheresis and field flow fractionation is described, for example, in U. S. Pat. App. Pub. No. 2004/0011651, incorporated herein by reference in its entirety. Using dielectrophoresis separation, adipose-derived regenerative cells can be manipulated to exploit differences in size, density, mass, and electrical properties, to separate different cells from other cells and to separate cells from non-cellular material such as processing reagents, enzymes, and other material such as collagen. A variety of cellular and non-cellular sizes would be separated, ranging from about 0.1 um (such as a collagen segment) to 30 um (such as a progenitor cell). In one embodiment, the present mechanism permits this further separation to occur within a sterile, closed fluid pathway that is fully integrated with prior and subsequent steps in cell isolation from the donor and delivery to the patient.

Thus, the composition derived from disaggregation of adipose tissue performed for the production of cells for subsequent clinical or therapeutic use as described, e.g., in U.S. application Ser. No. 10/316,127 and U.S. application Ser. No. 10/877,822, is introduced into and passed through a chamber in a closed, sterile fluid pathway or conduit. Material may be introduced into the conduit of the present invention prior to or following concentration. Concentration may be performed both prior to and following passage of cells through the separation conduit of the present invention. Electrodes surrounding the conduit apply at least one alternating electrical signal at different phases thereby creating a traveling electrical field which may also be spatially inhomogeneous. As a result the cells experience a field frequency and magnitude dependent lateral dielectrophoretic force as well as a rotational dielectrophretic force which reflects properties intrinsic to the cell, such as dielectric permittivity and electrical conductivity. Thus, cells with different properties will experience different forces within the field and may, therefore, be separated from one another. Combining such forces with the gravitational force and the forces experienced by fluid flow within the chamber creates a mechanism by which balancing and tuning one or more key parameters (including, but not limited to fluid flow rate, medium composition, and the magnitude, frequency, and phase of the signal received by the electrode) allows different cell types to be separated from one another and for cells to be separated from materials such as collagen fragments, cell aggregates, lipid, and enzymes and other reagents used in tissue processing and disaggregation.

For example, application of electrical field and flow parameters cause the cells to be attracted to and retained in proximity to the electrode while the medium passes freely through the conduit along with reagents, lipid, and/or collagen. This approach may be used to reduce the quantity of unwanted materials in the composition. Once an intended quantity of unwanted materials is removed the field properties may be changed, for example, the electrical signal may be turned off, thereby reducing, eliminating, or even reversing the attractive force applied to the cells such that they may now freely pass through the conduit and collected for subsequent delivery to a recipient. In the same way, the charged nature of collagen fragments may be exploited such that by applying a different set of flow and field parameters unwanted material, for example collagen, is retained in proximity to the electrode and the cells of the composition pass freely through the conduit for collection and subsequent delivery to a recipient.

In another embodiment, flow and field parameters are adjusted such that different cell types within the composition are separated from one another by differential attraction to or repulsion from an electrode in combination with separation according to their sedimentation rate, size, mass, and/or density. In this way the stem cells may be separated from other cells of the composition.

In embodiments the conduit is contained within a closed, sterile fluid pathway that is fully integrated with, or that may be connected in a functionally closed, sterile fashion with, components earlier in tissue processing—for example, tissue disaggregation—or those later in processing, for example, delivery of cells to the recipient. In this embodiment the conduit comprises part of a sterile, single use, disposable device. The electrode(s) can be included within this disposable set or may be included within a reusable, preferably automated device. In one embodiment the dielectrophoretic field/flow conduit is part of a sterile, closed system in which different components of the system are placed within an automated device. Thus, the components in which tissue disaggregation occurs or in which centrifugation or other post-disaggregation procedures occur are contiguous with the conduit used for dielectrophoretic/flow separation. Further, the port from which the desired output from the dielectrophoretic flow conduit is collected is contiguous with a chamber from which cells may be retrieved in a closed or functionally closed, sterile fashion for delivery to the recipient. In another embodiment a fluid path segment of a sterile, closed system including a flow conduit, canister, bag, or fluid path portion, is placed into an interface containing a mechanism such as a slot, sheath, ring, clamp or bracket in which the dielectrophoretic forces are induced externally by an electrode array through the walls of the closed fluid path segment. The desired separated output from said fluid path segment can then be further processed prior to delivery to the recipient, or, alternatively, be delivered to the recipient without further processing. In another embodiment the conduit of the present invention is initially non-contiguous with other components but is inserted into the system or otherwise made contiguous with it. In embodiments the means by which the conduit is made contiguous with the remainder of the system maintains a closed or functionally closed sterile fluid pathway. A sterile connecting device such as that manufactured by the Terumo Corporation is one means by which this connection may be made. In another embodiment, a sterile, closed system includes multiple pathways in which dielectrophoretic forces are induced sequentially or separately in parallel to segregate particles as part of the processing steps.

### (v) Preferential Lysis

Pulsed electric fields have been used to inactivate cells based on size. Larger cells, e.g., monocytes, can be preferentially porated and destroyed, while stem cells are preserved. (See, e.g., Craiu, et al., 2005, Blood 105(5):2235-2238, and Eppich, et al., 2000, Nature Biotechnology 18:882-887, incorporated herein by reference.) A flowing pulsed electric field apparatus can be used in conjunction with or as part of the sample processing device described herein, for example in a step taking place after sample washing and/or disaggregation.

Altering osmolarity has also been described to cause preferential cell lysis. Therefore, methods of preferentially removing certain cells by changing osmolarity conditions are contemplated. Furthermore, the density of cells can be preferentially changed, and the cells subsequently separated based on density. For example, increased separation efficiency in a bottom and top (BAT) procedure was reported when blood was collected in a hyperosmolar anticoagulant (Knutson, et al., 1999, Transfusion Science 21: 185-191).

### (vi) Positive and Negative Selection

A number of methods for enriching or negatively selecting cell subpopulations applicable to the methods of the present invention have been described in the art. For example, monoclonal antibodies and immunobeads (e.g., immunomagnetic beads), FACS, or antibody-coated columns, can be used to selectively separate certain cells from a population based on the cells' expression markers. Protein binding methods using, e.g., “Interfacial Biomaterials,” site-specific biological delivery (Affinergy, Inc.) can also be used.

In a magnetic immunobead strategy, monoclonal antibodies specific for a certain marker can be bound to the cells, then magnetic beads coated with a second antibody that recognizes the monoclonal antibody added. The target cell subpopulation can then be removed from the rest of the sample using a magnet. Antibody separation methods have been described, e.g., by Wang, et al., 1992, Bone Marrow Transplant. 9(5):319-23; Shimazaki, et al., 1998, Blood 72(4):1248-54, and; Rambaldi, et al., 1998, Blood 91(6):2189-2196.

### (vii) Filtration

A variety of filters available for separating cells based on size can be used, either alone or in conjunction with another separation method disclosed herein. Filtration methods and filters for separating cells from fluid samples are described at length in, e.g., U.S. Pat. App. Pub. Nos. 2004/0142463, incorporated herein by reference in its entirety.

For example, cord blood filters, such as the StemQuick™ E Cord Blood Filter, can be used to deplete the sample of red blood cells, platelets, and granulocytes. See, e.g., Eichler, et al., 2003, Stem Cells 21:208-216. Unwanted cells can also be removed by cryogel/hydrogel filtration, cross-flow filtration, coated porous scaffolds, apatite filtration, size exclusion filters, porous polyurethane membrane filters, and leukocyte depletion filters. These filters can be incorporated into the processing device at any point and in multiple positions, as desired.

III. Evaluating the Effectiveness of Manipulations

Numerous methods known to those of skill in the art are available for evaluating the effectiveness of the manipulations described in the present invention. For example, pelleted, nucleated ADCs can be manually counted using fluorescence labeling. A number of blood cell types and parameters can be quantified by automatic cell counting using a Coulter Hematology Analyzer.

Colony forming units (CFUs), representative of adherent cell colonies, can also be determined by methods described herein and widely known to those of skill in the art. Macromolecules, e.g., lipids, collagen and extracellular hemoglobin can also be assayed according to methods known in the art.

Electrodes can be inserted into the sample to obtain electrical impedance readings indicative of the state of the sample and how that sample should be further treated. For example, impedance readings can be taken during digestion with a disaggregant such as collagenase, and subsequent rinsing, to determine the extent of disaggregation.

In embodiments, the electrodes are used to evaluate the sample or any other fluid, e.g., waste or rinse solution. The results can be interpreted based on a correlation between the impedance measurements and the state of the sample as observed by other methods, e.g., sample visualization. Not only absolute values, but changes in values (e.g., as represented by the slope of the curve obtained when the values are plotted over time), and specific variations in values over time that correlate with the sample state as observed by other methods, could be used to derive algorithms to aid in evaluating the state of the sample. Besides degree of disaggregation, sample characteristics that can be evaluated by electrical impedance include, but are not limited to, degree of clumping and presence of desired or undesired substances or particles.

It is understood that methods for evaluation and combinations thereof can be used for directly assessing the effectiveness of a processing treatment but also for deriving algorithms applicable to assessing effectiveness, e.g., through programming of the processing device. The electrodes could be inserted in various parts of the system depending on the fluid to be evaluated, e.g., into container 20 to evaluate the sample directly, or into the output tube for container 20 to evaluate the exiting fluid. As known to those of skill in the art, the electrodes preferably consist of an inert material, e.g., gold.

Further evaluation of the sample at any point during processing can be made based on changes in optical density, color change, flow cytometry, apoptosis assays, cytokine assays, and in vitro and in vivo differentiation assays, all described in the literature and known to those of skill in the art.

Further chemical, biological or physical manipulation of the cells may also be initiated by reconfiguring the interconnections of the disposable sets of the existing system, re-programming the processing device of the existing system, by providing different or additional containers and/or chambers for the existing system, by transporting the cells to a one or more additional systems or devices and/or any combinations thereof. For example, the system can be reconfigured by any of the means described above such that the regenerative cells obtained using the system may be subject to one or more of the following: cell expansion (of one or more regenerative cell types) and cell maintenance (including cell sheet rinsing and media changing); sub-culturing; cell seeding; transient transfection (including seeding of transfected cells from bulk supply); harvesting (including enzymatic, non-enzymatic harvesting and harvesting by mechanical scraping); measuring cell viability; cell plating (e.g., on microtiter plates, including picking cells from individual wells for expansion, expansion of cells into fresh wells); high throughput screening; cell therapy applications; gene therapy applications; tissue engineering applications; therapeutic protein applications; viral vaccine applications; harvest of regenerative cells or supernatant for banking or screening, measurement of cell growth, lysis, inoculation, infection or induction; generation of cells lines (including hybridoma cells); culture of cells for permeability studies; cells for RNAi and viral resistance studies; cells for knock-out and transgenic animal studies; affinity purification studies; structural biology applications; assay development and protein engineering applications.

In all of the foregoing embodiments, at least a portion of the separated and concentrated regenerative cells may be cryopreserved, as described in U.S. patent application Ser. No. 10/242,094, entitled PRESERVATION OF NON EMBRYONIC CELLS FROM NON HEMATOPOIETIC TISSUES, filed Sep. 12, 2002, which claims the benefit of U.S. Provisional Patent Application 60/322,070 filed Sep. 14, 2001, which is commonly assigned, and the contents of which in their entireties are expressly incorporated herein by reference.

The following examples are provided to demonstrate particular situations and settings in which this technology may be applied and are not intended to restrict the scope of the invention and the claims included in this disclosure.

Citation of documents herein is not intended as an admission that any of the documents cited herein is pertinent prior art, or an admission that the cited documents are considered material to the patentability of the claims of the present application. All statements as to the date or representations as to the contents of these documents are based on the information available to the applicant and do not constitute any admission as to the correctness of the dates or contents of these documents.

### EXAMPLES

The present invention is further illustrated by the following examples, which should not be construed as limiting in any way.

### Example I

### Differential Adherence of Cells to a Solid Phase Structure

This example demonstrates the differential adherence of cells to a solid phase structure which allows for selective enrichment of a target or effector regenerative cell population.

### 1. Short Term Adhesion to Plastic

Fresh human ADC were plated onto tissue culture wells in regular medium (DMEM/F12+FCS) or saline+5% FCS and allowed to sit for different time periods. Non-adherent cells were rinsed off and the medium was replaced with regular medium to allow attached cells to proliferate.

### Results

Less than 10% of the cells adhered to the plastic. Cells that had been allowed as little as 1 hour of adherence time in regular medium showed growth to confluence within one week. Longer adherence time did not substantially improve cell growth. Cells plated in saline+5% FCS showed substantially poorer adhesion and growth although some growth was evident in the 1 hour adhesion wells. Five hour adhesion in saline+FCS showed good subsequent cell growth.

The results demonstrate quantitative adherence of cells to plastic in as little as an hour. Adhesion to other 2D surfaces including demineralized bone and apatite-coated poly-lactide surfaces, quantitation of cell enrichment and examination of shorter incubation times (<60 mins) will also be studied.

### 2. Short Term Adhesion to 3D Scaffolds.

In a second study fresh human ADCs were loaded onto an apatite-coated polylactide scaffold. The scaffold was placed at the base of a syringe and cells were passed through it five times over approximately 2 minutes. The scaffold was then allowed to sit for 1 hour after which the cells that did not bind were passed through an additional five times. The scaffolds were then rinsed, fixed, and stained, and viewed by conventional light microscopy to examine cell adhesion. The cells appeared as dark blue/black dots within the scaffold. Cells penetrated into the scaffold, not just the surface.

### 3. Medium Term (Four Hour) Adhesion to 3D Scaffolds

The growth characteristics of one hour adherent cells suggests that they are ADSCs. Thus, they likely have osteogenic capacity. In addition to other functions. An apatite-coated polylactide scaffold was loaded for four hours with fresh human adipose-derived cells (no prior culture). Cells were not perfusion loaded; rather the cells were placed on the scaffold and entered the scaffold by gravity over four hours. After four hours the scaffold was rinsed several times and then placed in DMEM/F12 culture medium with FCS (no osteo-inducer). Scaffolds were harvested after 7 days of culture and showed substantial areas of scaffold covered in sheets of cells. Similar results were achieved with a Bicalcium phosphate (BCP) scaffold. Qualitatively, the apatite-coated PLA scaffolds appeared to have more cells than the BCP scaffolds.

### Summary

Adherence to solid phase matrices, e.g., tissue culture plastic, may remove approximately 90% of non target regenerative cell populations in as little as one hour or less. In addition, regenerative cells can be seeded onto a scaffold (e.g., an osteoconductive scaffold) in one to four hours and, in the four hour loading, achieve cell proliferation on the scaffold in vitro.

### Example II

### Separation by Density Gradient Centrifugation

Density gradient centrifugation was used to separate the mononuclear cell fraction from adipose-tissue-derived regenerative cells.

Human adipose-derived cells were obtained following informed consent from individuals undergoing elective cosmetic liposuction. The tissue was collected through vacuum liposuction and rinsed with saline to remove excess blood. The ADC were obtained, as described herein and in, e.g., U. S. Pat. Pub. No. 2005/0084961, by enzymatic digestion and centrifugation, and the pelleted nucleated fraction isolated.

Lymphocyte Separatium Medium (LMS—Mediatech) was used to reduce the number of red blood cells in the sample. The LSM was allowed to equilibrate to room temperature and thoroughly mixed by gently inverting the bottle. The cell suspension was diluted to approximately 5 to 10 million cells/ml in sterile PBS (Ca⁺⁺ and Mg⁺⁺ free). A gradient of between 1:2 and 1:3 (LSM:cell suspension) was used. The appropriate volume of LSM was aseptically transferred to a sterile 50 ml conical centrifuge tube. The cell suspension was then carefully layered over the LSM in a sterile 50 ml conical tube, creating a sharp LSM-cell suspension interface. The tube was centrifuged at 400×g at room temperature for 20 minutes, and the top layer of PBS was aspirated to within 2-3 mm above the mononuclear cell layer.

The mononuclear cell layer was removed with as little of the LSM layer below it as possible, and the mononuclear cell layer transferred to a sterile centrifuge tube. A 3× volume of PBS (Ca⁺⁺ and Mg⁺⁺ free) was added, and the tube centrifuged for 5 minutes at room temperature at 400×g. The cells were washed again with PBS (Ca++ and Mg++ free) and resuspended in PBS (Ca⁺⁺ and Mg⁺⁺ free). Both fractioned and non-fractioned samples were stained with the antibodies shown in Table 1 below and analyzed for CFU-F content as well. For CFU-F, 1000 and 10000 cells were plated in 10 mm Petri dishes (6 replicates) and cultured for 14 days in DME/F12 medium supplemented with 10% fetal bovine serum and 1% antibiotic/antimycotic solution. The cells were fed once weekly with complete media change. To calculate the average number of colonies, only counts from 4 replicates were used (the lowest and highest counts were the ones left out to calculate the average).

For flow cytometric studies, the cells were resuspended in PBS (Ca⁺⁺ and Mg⁺⁺ free) supplemented with 1 mM EDTA, 25 mM HEPES, 1% fetal bovine serum and 10 U/ml DNAse I (Sorting Buffer).

No reduction in the number of CFU-F was observed when the fractioned and non-fractioned samples were compared (Table 1). Furthermore, FACS analysis showed no substantial difference in the scatter properties (FSC) of fixed cells between fractioned and non-fractioned samples (data not shown). These results indicate that blood cells can be separated from the sample in a density gradient without affecting regenerative cell viability.

TABLE 1

| | Total Sample | Fractioned Sample |
|---|---|---|
| CFU-F per 1000 cells plated | 7.5 | 12.25 |
| CFU-F per 10000 cells plated | 78.25 | 83.75 |

Furthermore, FACS analysis showed no substantial differences in the percentages of certain marker-expressing ADC subpopulations observed between the fractioned and non-fractioned samples. Preservation of the observed subpopulations during the fractioning procedure is further indication that the regenerative cells can be manipulated using density gradient centrifugation to remove blood cells.

### Example III

### Concentration of Cells by Continuous Flow Centrifugation

Continuous flow centrifugation was demonstrated to effectively concentrate cells. A continuous flow apparatus was constructed on the flat surface of a spinning disc. A cylindrical tube was mounted in roughly a U-shape on the flat surface of the disc. The parallel arms of the U were made using narrow plastic tubing (⅛ inch inside diameter, DEHP free PVC). The “bend” of the U was located at the perimeter of the disc, which was about 13 inches in diameter. At the bend, the connection between the inlet and outlet tubings was provided by an L-shaped mechanical port septum (SmartSite™, Alaris), connected to the tubing such that one of the equal-length arms of the L was attached to the inlet tubing and the other attached to the outlet tubing. The elbow of the L was oriented below the level of the tubing, and below the plane of the disc surface. Thus, each of the arms was oriented at a 45-degree angle to the surface of the disc. The elbow of the mechanical port septum forced fluid downwards as well as around the bend, and formed a trap for catching denser particles entering the bend during centrifugation.

To test the ability of this device to concentrate cells, fresh whole blood mixed with saline was pumped, using a peristaltic pump, through a stationary housing surrounding the centrifuge shaft in the center of the disc. The disc was spun at approximately 5000 rpm. The sample traveled outward through the tubing toward the perimeter of the disc, to the elbow bend where the denser particles (e.g., blood cells and stem cells) became trapped. The fluid then exited the device by traveling inward toward the center of the disc and through the housing.

The hematocrit in the starting fluid sample (saline mixed with fresh whole blood) was compared with that in the exiting (waste) fluid sample. The starting sample contained 21.1% hematocrit, while the waste contained 0.3%. This result indicated that the device successfully retained most of the cells.

### Example IV

### Evaluation of Disaggregation by Electrical Impedance Analysis

A study of electrical impedance change, in the collection chamber, during rinsing and digestion was performed using an electrode device as described above. It was noted that the slope of the plotted impedance measurements (in ohms) varied significantly during the time course studied (data not shown). This indicated that a correlation could be made between an observed degree of completion of digestion, disaggregation, or washing, and electrical impedance.

The observed correlation can be used in programming the processing device to sense a desired degree of processing completeness or purity in the sample, thereby prompting, e.g., continuation of the current processing step or progression to the next step. In further embodiments, multiple methods of evaluation are used for optimization of processing.

Each of the publications and patents cited herein are hereby incorporated by reference in their entireties. Those skilled in the art will recognize, or be able to ascertain using no more than routine experimentation, many equivalents to the specific embodiments of the invention described herein. Such equivalents are intended to be encompassed by the following claims.

What is claimed is:

1. A method for processing regenerative cells from adipose tissue comprising:
(a) introducing adipose tissue into a tissue collection container of a device configured to harvest regenerative cells from adipose tissue, while maintaining a closed, sterile fluid/tissue pathway;
(b) reducing the presence of free lipids and peripheral blood elements from said adipose tissue in the tissue collection container;
(c) disaggregating said adipose tissue in the tissue collection container to generate a cell suspension;
(d) separating the regenerative cells from the acellular component in the suspension;
(e) concentrating the regenerative cells; and
(f) manipulating the regenerative cells to obtain processed regenerative cells, wherein the manipulation is selected from the group consisting of exposure to hypoxic conditions, exposure to hyperoxic conditions, exposure to UV light, exposure to infrared light, exposure to ultrasonic stimulation, and exposure to electrical stimulation.

2. The method of claim 1, further comprising delivering the processed regenerative cells to a patient.

3. The method of claim 1, further comprising heating or cooling the regenerative cells.

4. The method of claim 1, wherein the separating comprises density gradient centrifugation, or continuous flow centrifugation, or both.

5. The method of claim 1, wherein the separating comprises adhering sample components to a solid phase surface.

6. The method of claim, wherein the solid phase surface is selected from the group consisting of tissue culture plastic, plastic beads, glass beads, and scaffolds or any combination thereof.

7. The method of claim 1, further comprising exposing the regenerative cells to an additive.

8. The method of claim 7, wherein the additive is selected from the group consisting of a tissue fragment, a growth factor, a cell differentiation factor, an immunosuppressive agent, an anti-apoptotic agent, and an anti-inflammatory agent.

9. The method of claim 1, further comprising combining the processed regenerative cells with a biologically compatible scaffold or carrier.

10. The method of claim 1, further comprising contacting the regenerative cells with DNAse I.

11. The method of claim 1, wherein the disaggregating comprises mechanical disaggregation of the adipose tissue.

12. The method of claim 11 wherein the mechanical disaggregation comprises ultrasonic disaggregation.

13. The method of claim 1, wherein the separating comprises filtration.

14. The method of claim 1, wherein the manipulation comprises exposure to hypoxic conditions comprising about 1% O₂.

15. The method of claim 1, wherein the regenerative cells comprise adipose-derived stem cells and endothelial progenitor cells.

16. The method of claim 9, wherein the biologically compatible scaffold or carrier is a resorbable scaffold.

17. The method of claim 9, wherein the biologically compatible scaffold or carrier is selected from the group consisting of a hyaluronon-based scaffold, an apatite coated scaffold, a hydrogel, and a collagen gel.

18. The method of claim 1, further comprising formulating the processed regenerative cells for injection.

19. The method of claim 1, wherein the degree of disaggregation is determined.

20. The method of claim 19, wherein the degree of disaggregation is determined by measuring current flow through the cell suspension, the optical density of the cell suspension, a color change in the cell suspension, or a color change in a waste solution generated during processing.
